(19) United States
(12) Patent Application Publication
(10) Pub. No.: US 2006/0182824 A1
(43) Pub. Date: Aug. 17, 2006

(54) Use of galactomannans as an active cosmetic agent

(76) Inventors: Caroline Lucas, Boulogne-Billancourt (FR); Frederique Poirier, Clamart (FR); Celine LaPerdrix, Guyancourt (FR)

Correspondence Address: BACHMAN & LAPOINTE, P.C., 900 CHAPEL STREET, SUITE 1201, NEW HAVEN, CT, 06510, US

(21) Appl. No.: 11/341,106

(22) Filed: Jan. 27, 2006

(30) Foreign Application Priority Data
Jan. 28, 2005 (FR) 0500889

(51) Int. Cl. A61K 36/48; A61K 31/715

(52) U.S. Cl. 424/757; 514/54

(57) ABSTRACT

This invention relates to a method for increasing the adhesion of basal keratinocytes to the Dermoepidermal Junction (DEJ) in a subject, comprising administrating an effective amount of a cosmetic or dermatological composition comprising galactomannans on the skin of the subject, in particular for preventing and/or treating skin aging.

### RELATED APPLICATION

This application is based on French Patent Application No. 05/00889, filed Jan. 28, 2005.

### FIELD OF THE INVENTION

This invention relates to a new use of galactomannans, alone or in combination, as an active agent enabling the expression of integrins α3β1 and α2β1, type V laminin and type VII collagen in keratinocytes to be increased, which expression increase enables the harmful effects of skin aging to be better counteracted

### BACKGROUND

The basal membrane of the skin or Dermoepidermal Junction (DEJ) corresponds to the anatomical zone between the basal cells of the epidermis and the more superficial layers of the dermis. This is a zone of adhesion between the epidermis and the dermis, providing control of small molecule filtration and the maintenance of adjacent cells (DAMOUR et al., Le vieillissement cutané, Ed. Flash Média, 1988).

The DEJ includes specific attachment complexes, called hemidesmosomes, the function of which is to provide the link between basal keratinocytes of the epidermis and the underlying basal membrane (KELLY, J. Cell. Biol., vol. 28, p. 61-73, 1966).

In addition to ensuring compartmentalisation, the DEJ plays a very important role both mechanically, as it enables solid anchoring of the epidermis, and biologically, as it is involved in cell signalling by means of proteins of the integrin family.

Integrins are transmembrane glycoproteins located on numerous cells such as keratinocytes in contact with the DEJ. These integrins have an extracellular portion enabling the recognition of characteristic proteins of the DEJ.

Among the components of the DEJ binding to the integrins, two proteins in particular, which play a fundamental role therein, can be cited:
type IV collagen, present in all basal membranes, synthetised by keratinocytes and fibroblasts. The protomers assemble to form the molecular framework from lamina densa which provides good mechanical stability and acts as a “screen” for the other proteins of the basal membrane, including anchoring proteins. One of the main anchorage protein corresponds to type VII collagen protomer, which assemble one each other to form anchorage fibrils implicated in dermoepidermal cohesion.
type V laminin, which is a constitutive protein of basal membranes, binds covalently to hemidesmosomes to form anchoring filaments.

These two proteins, by means of membrane receptors corresponding to integrin α3β1 for type V laminin and integrin α2β1 for type IV collagen, enable:
the basal keratinocytes to adhere to the support according to a well-defined orientation, and thus to structure the DEJ by promoting the anchorage of said basal keratinocytes;
tissue signals (such as keratinocyte proliferation and differentiation signals) and cell signals to be transmitted from the dermis to the epidermis.

These integrins constitute actual interfaces for communication between the inside and the outside of the cell, but also well beyond the basal layer since they contribute to better communication between the two main compartments of the skin, the dermis and the epidermis, by promoting cell adhesion.

As the skin ages, a flattening and thinning of the DEJ is observed. The adhesive properties of the epidermis are then reduced owing to a reduction in the expression of integrins specifically involved in the adhesion of basal keratinocytes (LEVARLET et al., J. Invest. Dermatol., vol. 3, p. 172-9, 1988). All of these modifications result in decreased communication between the compartments, probably contributing to dermoepidermal attachment alterations.

### SUMMARY OF THE INVENTION

The invention relates to a method for increasing the adhesion of basal keratinocytes to the Dermoepidermal Junction (DEJ) in a subject, comprising administrating an effective amount of a cosmetic or dermatological composition comprising galactomannans on the skin of said subject.

In various embodiments, said galactomannans correspond to polysaccharides from plant seeds made up of a linear chain β-D-mannopyranoses linked at β(1-4) and substituted by alpha-D-galactopyranose units at α(1-6), preferably from Fabaceae seeds, and most preferably from tara seeds (Caesalpinia spinosa (Molina) Kuntze).

In other embodiments, the galactomannan concentration in the composition is between 0.01 and 3% (w/w) of the total weight of the composition, preferably between 0.05 and 2% (w/w), by example between 0.05 and 1% (w/w) or between 0.05 and 0.5% (w/w), and most preferably between 0.05 and 0.4% (w/w) of the total weight of the composition.

### BRIEF DESCRIPTION OF THE DRAWING

FIG. 1 shows the labelling corresponding to the integrin α2β1 observed (magnified 20×) for keratinocytes cultivated on an uncoated support (FIG. 1A) or coated with 7 μg/cm² of type V laminin (FIG. 1B) and in the absence of a treatment with tara gum.

FIG. 2 shows the labelling corresponding to the integrin α2β1 observed (magnified 20×) for keratinocytes cultivated on an uncoated support (FIG. 2A) or coated with 7 μg/cm² of type V laminin (FIG. 2B) and treated with tara gum at a concentration of 10⁻²% in the culture medium.

### DETAILED DESCRIPTION

The aim of this invention is specifically to provide means making it possible to increase specifically the adhesion of basal keratinocytes to the major proteins of the DEJ, which are type V laminin and type IV collagen, so as to overcome the communication disturbances between the dermis and the epidermis in skin aging.

This aim is achieved by cosmetic or dermatological compositions for skin care and more specifically for fighting against skin aging, including, as the active agent, galactomannans from plant seeds made up of a linear chain of β-D-mannopyranoses linked at β(1-4) and substituted by alpha-D-galactopyranose units at α(1-6) and more specifically galactomannans of tara gum, from the Caesalpinia spinosa (Mol.) Kuntze seed.

Indeed, in addition to their rheologic properties, the inventors have demonstrated that a “small” concentration of such galactomannans, in particular galactomannans from tara gum, was associated with highly advantageous biological properties against skin aging, namely improvement of the adhesion of basal keratinocytes to the proteins of the DEJ by the promotion of increased expression of integrins α3β1 and α2β1, and also of type V laminin and type VII collagen.

Galactomannans come from the endosperm of the seeds. After the removal of the shell and the germ, the endosmperm is grind and the powder obtained is commonly called “gum”. The galactomannan content of these gums is high and can reach 80% by weight for tara gum. The main gums used industrially are guar gum, carob gum and, recently, tara gum. The galactomannan content of these gums is high and can reach 80% by weight for tara gum. Galactomannans are widely used in the food and pharmaceutical industry as natural thickeners. Galactomannans are thus used in cosmetic or pharmaceutical compositions in which they act as thickening agents.

Consequently, a first objective of the invention corresponds to a method for increasing the adhesion of basal keratinocytes to the Dermoepidermal Junction (DEJ) in a subject, comprising administrating an effective amount of a cosmetic or dermatological composition comprising galactomannans on the skin of said subject.

In fact, said composition enables to increase the expression of integrins α3β1 and α2β1 in basal keratinocytes, and also the expression of type V laminine and type VII collagen by keratinocytes, which correspond to key proteins for the adhesion of basal keratinocytes to the DEJ.

The term galactomannans refers to polysaccharides from plant seeds, which are made up of a linear chain of β-D-mannopyranoses linked at β(1-4) and substituted by alpha-D-galactopyranose units at α(1-6).

The term subject refers to a mammal, and preferably to a human.

An effective amount of said dermatological or cosmetic composition can be simply determined by one of skill in the art. As an example, such effective amount can correspond to an administration of 0.05 to 100 mg/cm² of said dermatological or cosmetic composition on the skin of said subject, preferably of 0.1 to 10 mg/cm², and most preferably from 0.5 to 5 mg/cm².

Advantageously, said administrating step is repeated every day for more than one week, preferably for more than two weeks, and most preferably for more than one month.

In plant seeds, which are the origin of the galactomannans used in the compositions of the invention, the galactomannans constitute an extracellular deposition of endosperm and serve as energy reserves used during germination. As examples of plant seeds that can be used in the compositions according to the invention, the following can be cited: Fabaceae seeds, such as guar seeds (Cyamopsis tetragonoloba (L.) Taubert), carob seeds (Ceratonia siliqua L.), fenugreek seeds (Trigonella foenum graecum L.), honey-locust seeds (Gleditsia triacanthos L.) and tara seeds (Caesalpinia spinosa (Molina) Kuntze). The seed used is preferably a tara seed (Caesalpinia spinosa (Molina) Kuntze).

The galactomannans used are advantageously in “gum” form. As examples of such gums that can be used in the compositions of the invention, the following can be cited: guar gum, carob gum, fenugreek gum, honey-locust gum or tara gum. The gum used is preferably tara gum.

Tara or Caesalpinia spinosa (Molina) Kuntze (Caesalpiniacae)—syn: C. tinctoria (H.B K) Bentham ex Reiche: it is a small thorny tree from 2 to 5 m high, with compound bipinnate leaves and coriaceous, sessile leaflets, of dark green colour on the upper surface and lighter on the lower surface. The tree flowers in September, the flowers, gathered in dense clusters, are reddish yellow. The calyx comprises 5 sepals, and the corolla comprises 5 petals. The fruit is a reddish, oblong and glabrous pod, containing 8 to 10 black seeds. This shrub is native to northern South American (Peru, Bolivia, northern Chile), and is also found in Ecuador, Venezuela and in some North African countries. It is cultivated in most of these countries. Peru is the primary producer, with 80% of the world's production, i.e. an average of 19,000 tons of fruit per year. The pods are used as a source of tannins for the leather industry while the endosperm of the seed is used for the production of gum.

The galactomannans that can be used in the compositions according to the invention have a galactose to mannose ratio between 1:2 and 1:100, preferably between 1:2 and 1:10 and most preferably between 1:2 and 1:5. This degree of substitution is characteristic of the plant species. For example, this degree of substitution is 1:3 for tara gum.

Galactomannans can be used in a modified or unmodified form, such as hydrolysates obtained chemically or enzymatically. Such modifications in particular make it possible to modify the degree of polymerisation of the linear chain of galactomannans.

The degree of polymerisation of the linear chain of galactomannans is advantageously between 100 and 10,000 mannose units, and preferably between 1,000 and 10,000 mannose units.

The galactomannan concentration in the composition is advantageously between 0.01 and 3% of the total weight of the composition, preferably between 0.05 and 2%, more preferably between 0.05 and 1%, by example between 0.05 and 0.5%, and most preferably between 0.05 and 0.4%.

Advantageously, said composition is intended to increase the adhesion of the basal keratinocytes to the proteins of the DEJ, preferably to improve the transmission of tissue signals between the dermis and the epidermis.

Finally, said composition is advantageously intended to prevent and/or treat skin aging.

The compositions according to the invention can also include one or more formulation agents or active agents having a known and conventional use in cosmetic and/or dermatological compositions. As a non-limiting example, the following can be cited: softeners, dyes, film-forming agents, surfactants, perfumes, preservatives, emulsifiers, oils, glycols, sebum absorbers, vitamins, and so on.

The active agents are advantageously plant extracts.

The compositions according to the invention can be in any form known to a person skilled in the art in the field of cosmetics and dermatology with the only galenic restriction being application on facial or body skin. The compositions according to the invention are advantageously provided in the usual cosmetic forms, such as, in particular, a simple O/W or W/O emulsion, multiple emulsions or micro-emulsions, aqueous or hydroalcoholic gels or oils or lotions.

Other advantages and features of the invention will appear in the following examples, which in no way limit the scope of the invention.

### EXAMPLES

1) Modulation of the Expression of Integrins α3β1 and α2β1 in a Monolayer Culture of Normal Human Keratinocytes.

Protocol:

In a first “coating” step, culture slides with 4 chambers (FALCON) were coated either with mouse type V laminin (BD SCIENES), or with mouse type IV collagen (BD SCIENCES), at concentrations of 0.3 or 7 μg/cm², then incubated for 24 hours at 37° C.

The medium was then removed and the chambers were rinsed with the K-SFM culture medium (INVITROGEN).

Normal human keratinocytes (NHK) were then seeded (passage 3) at a rate of 7,000 cells/chamber in a supplemented K-SFM culture (INVITROGEN). The cells were then placed in an incubator at 37° C., 5% CO₂ and 95% saturated humidity for 6 days.

The culture medium was then removed and replaced by a new culture medium supplemented with tara gum at concentrations of 10⁻²%, 10⁻³% or 10⁻⁴% (p/v). A control chamber was supplemented with a new culture medium not supplemented with tara gum. The tara gum used for the experiment was food-grade and non-hydrolysed (EXANDAL). The tara gum was solubilised extemporanously to the experiment in water at 60° C., for one hour and at a concentration of 0.1%.

The cells were then placed in an incubator under the conditions described above, for 3 days.

The culture slides were then taken down. The cells were then fixed with a 4% formaldehyde solution (SIGMA) in PBS (Phosphate Buffer Saline). The cells were then permeabilised with a 0.1% TRITON X-100@ solution (SIGMA).

The slides were then incubated with a saturation solution, including 5% BSA (SIGMA) in PBS, to block the non-specific sites.

A culture slide was then incubated with a 1% PBS BSA solution supplemented or not with a primary integrin α3β1 (mouse monoclonal IgG, TEBU-BIO) or α2β1 (mouse monoclonal IgG, TEBU-BIO) antibody.

The slides were then rinsed three times in a 1% PBS BSA solution. The slides were then incubated in the presence of a 1% PBS BSA solution supplemented with a secondary antibody (goat anti-mouse IgG, rhodamine conjugated, TEBU-BIO) at 1/500^{th}.

The slides were then rinsed three times in a 1% PBS BSA solution and mounted on a plate using a drop of mounting medium (Fluorescent Mounting Medium, DAKOCYTOMATION).

The slides were then observed using a microscope combined with a fluorescence system (OLYMPUS). An adapted filter enabled the excitation wavelength of 550 nm to be selected (rhodamine).

Results:

Effect of Coating on Integrins Expression:

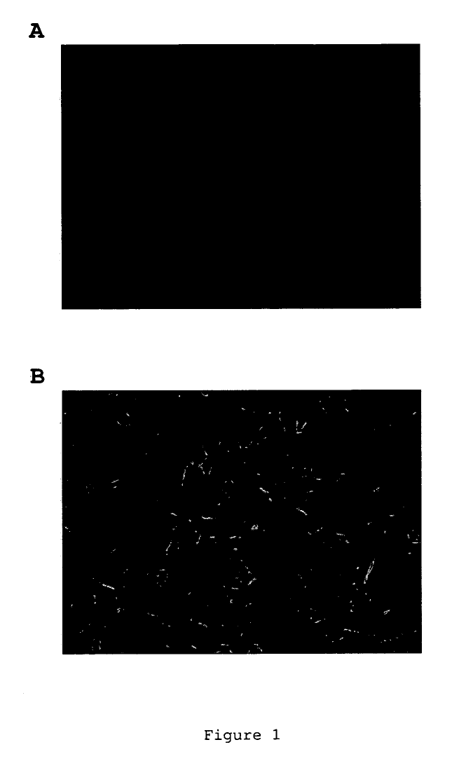

In the absence of a treatment with tara gum, the results obtained in the absence (FIG. 1A) and in the presence of a preliminary coating of the culture slides with type V laminin (FIG. 1B) show that said coating step causes an increase in the expression of integrin α2β1 by the keratinocytes. More generally, the results have shown that the preliminary “coating” of the culture slides with type V laminin or type IV collagen causes a simultaneous increase in the expression of integrins α3β1 and α2β1 by the keratinocytes.

Effect of the Tara Gum Treatment:

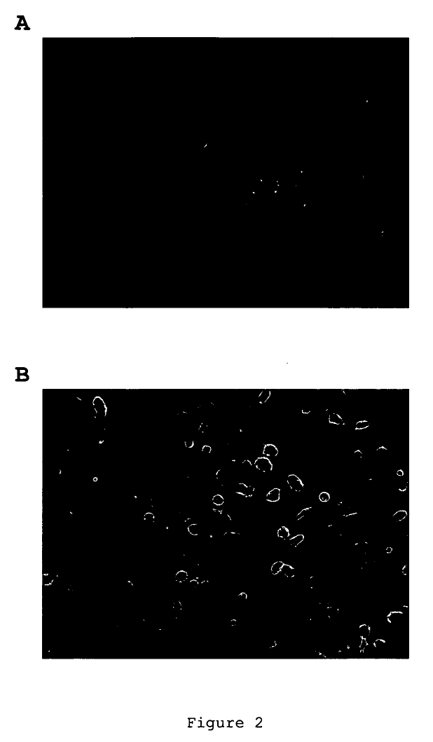

In the absence of coating, the results show that the treatment with tara gum causes an increase in the expression of integrin α2β1 by the keratinocytes (FIG. 2A versus FIG. 1A in the absence of tara gum). Such an increase in the expression of integrin α2β1 by the keratinocytes is also observed in the case of a preliminary coating with type V laminin (FIG. 2B versus FIG. 1B in the absence of tara gum). More generally, the results have shown that the treatment of keratinocytes with tara gum causes a dose-dependent increase in the expression of integrins α3β1 and α2β1 by the keratinocytes.

2) Modulation of the Expression of Type V Laminin and Type VII Collagen in a Monolayer Culture of Normal Human Keratinocytes:

Protocol:

Normal human keratinocytes (NHK) were cultured as described previously on non-coated dishes, and then placed in an incubator at 37° C., 5% CO₂ and 95% moisture for 1 day.

The culture medium was then removed and replaced by a new culture medium supplemented with tara gum at a concentration of 10⁻³% (p/v). A control chamber was supplemented with a new culture medium not supplemented with tara gum.

The cells were then placed in an incubator under the conditions described above, for 1 day.

Then, total RNA and proteins have been then extracted by TRI REAGENT (SIGMA) according to manufacturer's instructions.

Expression of type V laminin (beta3 chain) and type VII collagen (alpha1 subunit) mRNAs have been determined by quantitative RT-PCR using “light cycler system” (ROCHE) kit according to the manufacturer's instructions by using specific primers of Type V laminin (β3 chain; genebank number: L25541) and of Type VII collagen (alpha1 subunit; genebank number: L02870).

Expression of type V laminin and type VII collagen proteins have been determined by western blot on SDS PAGE gels with antibodies specific from type V laminin and type VII collagen (TEBU-BIO) detected by ECL techniques according to the manufacturer's instruction.

Results:

The results are summarized in table I.

| | |
|---|---|
| expression of specific | expression of specific |
| mRNAs in tara gum | proteins in tara gum |
| treated keratinocytes | treated keratinocytes |
| compared to control | compared to control |

| | type V | type VII | type V | type VII |
|---|---|---|---|---|
| | laminin | collagen | laminin | collagen |
| Tara gum | +28% | +48% | +14% | +34% |
| (0.1%) | | | | |

The results show that the treatment of keratinocytes with tara gum increases DEJ major proteins synthesis—i.e. type V laminin and type VII collagen—, and thus enables to reinforce this structure, which is implicated in cutaneous architecture.

3) Incorporation in a Cosmetic Formula:

Example of Cosmetic Composition of the Invention in the Form of an Emulsion:

| Compound | Proportion |
|---|---|
| Tara gum | 0.1% |
| Mixture of preservatives | 1.5% |
| Glycol propylene | 5.00% |
| Xanthan gum | 0.30% |
| Acrylic/acrylate copolymer | 0.50% |
| Stearic acid 100 OE | 3.00% |
| Sorbitan stearate | 2.00% |
| Sorbitan laurate 20 OE | 3.00% |
| Cetyl Stearyl alcohol | 1.50% |
| Beeswax | 1.00% |
| Wheat germ oil | 5.00% |
| Dimethicone | 2.00% |
| Cyclomethicone | 5.00% |
| Polyacrilamide gel | 2.00% |
| Perfume | 0.10% |
| Water | qsp 100% |

Example of Cosmetic Composition of the Invention in the Form of a Cream:

| Compound | Proportion |
|---|---|
| Tara gum | 0.1% |
| Preservatives | 1.5% |
| Chelating agent | 0.05% |
| Xanthan gum | 0.30% |
| Acid C18 | 2.50% |
| Acid C16 | 2.50% |
| Trilaurin | 1.00% |
| Shea butter | 0.05% |
| Tocopheryl acetate | 0.05% |
| Beta bisabolol | 0.05% |
| Wheat germ oil | 5.00% |
| Dimethicone | 3.00% |
| Cyclomethicone | 5.00% |
| Polyacrylic acid | 0.30% |
| TEA (triethanolamine) | 1.50% |
| Perfume | 0.10% |
| Water | qsp 100% |

1. A method for increasing the adhesion of basal keratinocytes to the Dermoepidermal Junction (DEJ) in a subject, comprising administrating an effective amount of a cosmetic or dermatological composition comprising galactomannans on the skin of said subject.

2. The method according to claim 1, wherein said administrating step comprises administrating a composite wherein said galactomannans correspond to polysaccharides from plant seeds made up of a linear chain β-D-mannopyranoses linked at β(1-4) and substituted by alpha-D-galactopyranose units at α(1-6).

3. The method according to claim 2, wherein said administrating step comprises administrating a composite wherein said plant seeds are Fabaceae seeds.

4. The method according to claim 3, wherein said administrating step comprises administrating a composite wherein said Fabaceae seeds are tara seeds (Caesalpinia spinosa (Molina) Kuntze).

5. The method according to claim 1, wherein said administrating step comprises administrating a composite wherein said galactomannans have a galactose to mannose ratio between 1:2 and 1:100.

6. The method according to claim 5, wherein said administrating step comprises administrating a composite wherein said galactomannans have a galactose to mannose ratio between 1:2 and 1:10.

7. The method according to claim 1, wherein said administrating step comprises administrating a composite wherein said galactomannans are in a modified or unmodified form.

8. The method according to claim 2, wherein said administrating step comprises administrating a composite wherein a degree of polymerisation of the linear chain of said galactomannans is between 100 and 10,000 mannose units.

9. The method according to claim 8, wherein said administrating step comprises administrating a composite wherein the degree of polymerisation of the linear chain of said galactomannans is between 1,000 and 10,000 mannose units.

10. The method according to claim 1, wherein said administrating step comprises administrating a composite wherein the galactomannans are present in a concentration in the composition between 0.01 and 3% (w/w) of a total weight of the composition.

11. The method according to claim 10, wherein said administrating step comprises administrating a composite wherein the galactomannan concentration in the composition is between 0.05 and 2% (w/w) of the total weight of the composition.

12. The method according to claim 11, wherein said administrating step comprises administrating a composite wherein the galactomannan concentration in the composition is between 0.05 and 1% (w/w) of the total weight of the composition.

13. The method according to claim 12, wherein said administrating step comprises administrating a composite wherein the galactomannan concentration in the composition is between 0.05 and 0.5% (w/w) of the total weight of the composition.

14. The method according to claim 13, wherein said administrating step comprises administrating a composite wherein the galactomannan concentration in the composition is between 0.05 and 0.4% (w/w) of the total weight of the composition.

15. The method according to claim 1, wherein said administrating step comprises administrating a composite wherein said composition further comprises one or more formulation agents or active agents selected from the group consisting of plant extracts, softeners, dyes, film-forming agents, surfactants, perfumes, preservatives, emulsifiers, oils, glycols, sebum absorbers, and vitamins.

16. The method according to claim 1, wherein said administrating step comprises administrating a composite wherein said composition is in a form selected from the group comprising simple O/W and W/O emulsion, multiple emulsions and micro-emulsions, aqueous and hydroalcoholic gels, oils and lotions.
